(19) United States
(12) Patent Application Publication
(10) Pub. No.: US 2008/0249028 A1
(43) Pub. Date: Oct. 9, 2008

(54) Thyrotropin-Releasing Hormone Analogs and Method of Use

(75) Inventor: LuGuang Luo, Seekonk, MA (US)

Correspondence Address: EDWARDS ANGELL PALMER & DODGE LLP, P.O. BOX 55874, BOSTON, MA, 02205, US

(73) Assignee: Roger Williams Hospital, Providence, RI (US)

(21) Appl. No.: 11/885,941

(22) PCT Filed: Mar. 8, 2006
(86) PCT No.: PCT/US06/08542

§ 371 Date: Apr. 10, 2008

Related U.S. Application Data
(60) Provisional application No. 60/660,175, filed on Mar. 9, 2005.

(51) Int. Cl. A61K 38/06; C12Q 1/02

(52) U.S. Cl. 514/18; 435/29

(57) ABSTRACT

The invention provides a method of modulating blood glucose levels by treating or preventing pancreas-related disorders with thyrotropin-releasing hormone (TRH) or a TRH derivative. Diabetes mellitus, pancreatic islet destruction, pancreatic beta cell malfunction, and hyperglycemia-related malfunction are preferably treated or prevented.

### RELATED APPLICATION

This application claims the benefit of U.S. provisional patent application Ser. No. 60/660,175, filed Mar. 9, 2005 (attorney docket no. 11259-61784P), the entire contents of which application are incorporated herein by this reference.

### BACKGROUND OF THE INVENTION

Thyrotropin-releasing hormone (TRH), which has been identified as L-pyroglutamyl-L-histidyl-L-prolineamide, is a small peptide that has been found in various cells of the body, mainly the neural cells of the central nervous system. The structure of TRH is as follows:

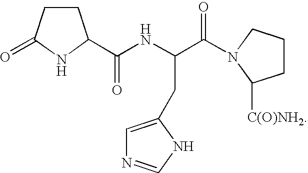

The right portion of the molecule is known to those skilled in the art as the “prolineamide”, “COOH-terminal” or “C-terminal” portion; the center portion of the molecule is known as the “histidyl” portion; and the left portion of the molecule is known as the pyroglutamyl , NH₂-terminal or “N-terminal” portion.

The function of TRH in various areas of the body is largely unknown. However, numerous studies have shown that administration of TRH to the peripheral or central nervous system induces blood glucose variation (Amir, S., et al. (1987) Brain Res. 435, 112-122; Ishiguro, T., et al. (1991) Neuroendocrinology 54, 1-6).

Endogenous TRH is known to act as either a neurotransmitter or a neuromodulator or both. A major percentage of this hormone is released from the hypothalamic nerve terminals in the median eminence to stimulate the secretion of thyroid stimulating hormone, the function for which TRH is named (Wu, P., and Jackson, I. M. (1988) Regul. Pept. 22, 347-360). TRH is also found in other areas of the central nervous system, and in tissues of the body such as the alimentary tract, pancreas, placenta and retina of the eye (Martino, E., et al. (1978) Proc. Natl. Acad. Sci. U.S.A., 75, 4265-4267.; Koivusalo, F., et al. (1979) Life Sci. 24, 1655-1658.; Morley, J. E. (1979) Life Sci. 25, 1539-1550.; Engler, D., (1982) J. Clin. Invest., 69, 1310-1320.; Aratan-Spire, S. et al., (1984) Acta Endocrinol. (Copenh.), 106, 102-108.; Leduque, P., et al. (1985) Regul. Pept. 10, 281-292.; Fuse, Y., et al. (1990) Endocrinology 127, 2501-2505.).

TRH and its analogs have been established as compounds useful for the treatment of neurologic damage, including brain trauma, spinal cord trauma, neurologic damage caused by a stroke, by anesthesia or by a drug overdose (U.S. Pat. No. 5,686,420). These injuries are treated by TRH or TRH analogs, wherein the reduction of secondary effects of the trauma can be carried out by antagonizing the actions of autodestructive biochemical substances. However, the use of TRH and its analogs in the treatment of neurologic damage does not logically lead one to conclude that TRH could be useful in the treatment of diseases outside the brain and spinal cord.

The major disadvantage of the use of TRH in the peripheral nervous system is that the hormone is metabolized very rapidly. Therefore, high doses or continuous infusions are necessary for effective treatment. The short plasma half-life (4-5 min.) is most likely due to rapid degradation of the peptide at both the COOH— and NH₂-terminals of the molecule. Cleavage of the pyroglutamyl moiety of TRH by peptidases causes formation of the metabolite cyclo-histidyl-proline-diketopiperazine. Deamidation of TRH results in the formation of the free acid TRH—OH.

Outside the central nervous system, TRH is expressed in pancreatic islet beta cells (Martino, E. et al. Proc. Natl. Acad. Sci. USA, 1978, 75: 4265-4267.; Koivusalo, F. et al. Life Sci., 1979, 24: 1655-1658.) and the pancreas has been shown to have TRH receptors (Yamada, M. et al. Life Sci. 2000, 66:1119-1125.; Luo, L. G. et al. http://journals.endocrinology.org/joe/fca/JOE05483.htm).

Mice with the TRH gene knockout have been shown to develop hyperglycemia. Further, it has been shown that thyroid hormone replacement does not reverse the hyperglycemia (Yamada, M. et al. Proc. Natl. Acad. Sci. USA., 1997, 94(20): 10862-10867.).

TRH and its analogues have been used for treating pancreatitis. In these studies, pain management, not the pancreatitis clinical course, was evaluated over a three hour period (Kiviniemi, H. et al. Acta. Chir. Scand. 1986, 152:43-47.). TRH activation of EGF receptors in a cultured pituitary cell line indicated TRH may have growth factor function (Wang, Y. et al. Mol. Endocrinol. 2000, 14:1328-1337.).

The ability of TRH or its analogs to treat diabetes mellitus has not been previously demonstrated. The injection of TRH into the rat brain has been reported to reduce blood glucose levels (Amir, S. et al. Brain Res. 1987, 435:112-122.; Ishikawa, Y. et al. Brain Res. 1990, 514:1-4). Also, TRH concentrations in rat pancreas decreased in streptozotocin-induced diabetes (Dolva, L. O. et al. J. Clin. Invest. 1983, 76(2):1867-1873.). Additionally, TRH has been proposed to be a potential target molecule of treatment for diabetes (Yamada, M. et al. Thyroid. 2003, 13:1111-1121.).

A number of peptidase-resistant analogs of TRH have been synthesized, mainly for research purposes. They were developed initially as antidepressants. Most of these analogs have been found to have centrally active effects such as endocrine, analeptic and autonomic effects. However, none of these have been proposed and/or tested to treat diabetes mellitus.

What is needed is a compound that is effective in modulating blood glucose levels by treating pancreas-related disorders such as diabetes mellitus, pancreatic islet destruction, pancreatic beta cell malfunction, and hyperglycemia-related malfunction. Especially sought is a compound that is effective in reducing blood glucose level in patients suffering from the loss of pancreatic endocrine beta cell function, without affecting thyroid function and without bringing about other undesirable side effects. Especially desirable analogs are those that are selective for TRH receptor type 1 activation, because the pancreas only expresses TRH receptor type 1, and that are not rapidly metabolized.

### SUMMARY OF THE INVENTION

In one aspect, the invention provides a method for modulating blood glucose in an animal, comprising administering to an animal in need thereof a therapeutically effective amount of a compound selected from the group consisting of thyrotropin releasing hormone (TRH), a TRH derivative, and a pharmaceutically acceptable salt, solvate, or hydrate of TRH or of a TRH derivative, to thereby modulate the blood glucose levels.

In another aspect, the invention provides a method of regenerating pancreatic beta cells and, hence, restoring and/or improving pancreatic function, comprising administering to an animal in need thereof an effective amount of a compound selected from the group consisting of TRH, a TRH derivative, and a pharmaceutically acceptable salt, solvate, or hydrate of TRH or of a TRH derivative.

In still another aspect, the invention provides a kit comprising a compound selected from the group consisting of TRH, a TRH derivative and a pharmaceutically acceptable salt, solvate, or hydrate of TRH or of a TRH derivative, together with instructions for treating a pancreas-related disorder.

In yet another aspect, the invention provides a packaged composition comprising a therapeutically effective amount of a compound selected from the group consisting of TRH, a TRH derivative and a pharmaceutically acceptable salt, solvate, or hydrate of TRH or of a TRH derivative and a pharmaceutically acceptable diluent or carrier, wherein the composition is formulated as a pharmaceutical composition for treatment of a pancreas-related disorder, and packaged with instructions for therapeutic use.

In still another aspect, the invention provides a method of identifying a TRH derivative that is capable of modulating blood glucose levels comprising contacting a pancreatic cell having impaired pancreatic function with a candidate TRH derivative, and determining if pancreatic function of the pancreatic cell is restored or improved, to thereby identify a TRH derivative that is capable of restoring or improving pancreatic function resulting in the modulation of blood glucose levels.

In another aspect, the invention provides a novel compound wherein the compound is a TRH derivative having formula (I):

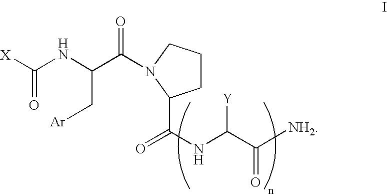

wherein:

X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;

Y is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, aryloxy, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, S(O)₂NR₂, or R;

wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;

and each X or Y may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;

Ar is selected from the following:

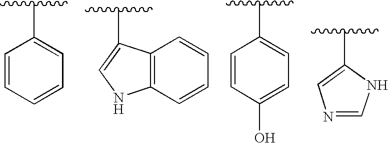

wherein each Ar group may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy; and n is an integer from 0-5.

In another aspect, the invention provides a method of inhibiting transplant rejection in a subject comprising administering to the subject TRH or a TRH derivative in an amount effective to inhibit transplant rejection in the subject.

### BRIEF DESCRIPTION OF THE DRAWINGS

FIG. 1 shows the effect of TRH on rat hyperglycemia.

FIG. 2 shows the effect of Glp-3-Me-His-Pro-NH₂ on rat hyperglycemia.

FIG. 3 shows the effect of Glu-His-Pro-NH₂ on rat hyperglycemia.

### DETAILED DESCRIPTION OF THE INVENTION

The instant invention is based, at least in part, on the discovery that thyrotropin-releasing hormone (TRH) and TRH derivatives/analogs are useful compounds for the modulation of blood glucose in an animal. The invention provides a method of modulating blood glucose by treating or preventing pancreas-related disorders with thyrotropin-releasing hormone TRH or TRH derivatives. Diabetes mellitus, pancreatic islet destruction, pancreatic beta cell malfunction, and hyperglycemia-related malfunction are pancreas-related disorders which can be treated by the instant invention.

The disorders are treated by administering an effective amount of TRH or a TRH derivative wherein the TRH or TRH derivative preferably reduces the secondary effects of the hyperglycemia by optimally antagonizing the actions of autodestructive biochemical substances, such as endogenous cytokines, without being rapidly metabolized. The TRH derivatives maintain some structural features of TRH, including the prolineamide and histidyl functionalities.

Although not wanting to be bound by the following hypothesis, it is believed that the TRH derivatives/analogs of the present invention increase calcium flow to facilitate insulin release from pancreatic beta cells. The analogs may also improve pancreatic beta cell recovery by blocking the actions of several injury factors including autoimmune over-reaction, releasing an apoptotic activating factor as a consequence of diabetes, and improving glucose uptake in muscles.

### Definitions

In order that the invention may be more readily understood, certain terms are defined and collected here for convenience.

The terms “analog” and “derivative” are used interchangeably. As used herein a “TRH analog” or a “TRH derivative” refers to a compound which retains chemical structures of TRH necessary for the desired functional activity of TRH (e.g., the prolineamide and histidyl functionalities), yet which also contains certain chemical structures which differ from that of TRH.

The term “alkyl” refers to the radical of saturated aliphatic groups, including straight-chain alkyl groups, and branched-chain alkyl groups. The term alkyl further includes alkyl groups, which can further include oxygen, nitrogen, sulfur or phosphorous atoms replacing one or more carbons of the hydrocarbon backbone, e.g. oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, and still more preferably 4 or fewer.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate.

The term “alkyl” also includes unsaturated aliphatic groups analogous in length and possible substitution to the alkyls described above, but that contain at least one double or triple bond respectively. An “alkylaryl” moiety is an alkyl substituted with an aryl (e.g., phenylmethyl (benzyl)).

The terms “alkoxy,” “aminoalkyl” and “thioalkoxy” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aralkyl” means an aryl group that is attached to another group by a (C₁-C₆)alkylene group. Aralkyl groups may be optionally substituted, either on the aryl portion of the aralkyl group or on the alkylene portion of the aralkyl group, with one or more substituents.

The term “aryl” as used herein, refers to the radical of aryl groups, including 5- and 6-membered single-ring aromatic groups that may include from zero to four heteroatoms (heteroaryl), for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like.

Those aryl groups having heteroatoms in the ring structure may also be referred to as “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “cyclyl” refers to a hydrocarbon 3-8 membered monocyclic or 7-14 membered bicyclic ring system having at least one non-aromatic ring, wherein the non-aromatic ring has some degree of unsaturation. Cyclyl groups may be optionally substituted with one or more substituents. In one embodiment, 0, 1, 2, 3, or 4 atoms of each ring of a cyclyl group may be substituted by a substituent. The term “cycloalkyl” refers to a hydrocarbon 3-8 membered monocyclic or 7-14 membered bicyclic ring system having at least one saturated ring. Cycloalkyl groups may be optionally substituted with one or more substituents. In one embodiment, 0, 1, 2, 3, or 4 atoms of each ring of a cycloalkyl group may be substituted by a substituent. Cycloalkyls can be further substituted, e.g., with the substituents described above. Preferred cyclyls and cycloalkyls have from 3-10 carbon atoms in their ring structure, and more preferably have 3, 4, 5, 6 or 7 carbons in the ring structure. Those cyclic groups having heteroatoms in the ring structure may also be referred to as “heterocyclyl,” “heterocycloalkyl” or “heteroaralkyl.” The aromatic ring can be substituted at one or more ring positions with such substituents as described above.

The terms “cyclyl” or “cycloalkyl” refer to the radical of two or more cyclic rings (e.g., cycloalkyls, cycloalkenyls, cycloalkynyls, aryls, heteroaryls, and/or heterocyclyls). In some cases, two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “mercapto” refers to a —SH group.

The term “sulfhydryl” or “thiol” means —SH.

The compounds of the invention encompass various isomeric forms. Such isomers include, e.g., stereoisomers, e.g., chiral compounds, e.g., diastereomers and enantiomers.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

Furthermore the indication of configuration across a carbon-carbon double bond can be “Z” referring to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

With respect to the nomenclature of a chiral center, the terms “d” and “I” configuration are as defined by the IUPAC Recommendations. As to the use of the terms, diastereomer, racemate, epimer and enantiomer, these will be used in their normal context to describe the stereochemistry of preparations.

Natural amino acids represented by the compounds utilized in the present invention are in the “I” configuration, unless otherwise designated. Unnatural or synthetic amino acids represented by the compounds utilized in the present invention are in the “d” configuration, unless otherwise designated.

Another aspect is a radiolabeled compound of any of the formulae delineated herein. Such compounds have one or more radioactive atoms (e.g., ³H, ²H, ¹⁴C, ¹³C, ³⁵S, ³²P, ¹²⁵I, ¹³¹I) introduced into the compound. Such compounds are useful for drug metabolism studies and diagnostics, as well as therapeutic applications.

The term “administration” or “administering” includes routes of introducing the TRH or TRH derivative compound(s) to a subject to perform their intended function. Examples of routes of administration which can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. The injection can be bolus or can be continuous infusion. Depending on the route of administration, the TRH or TRH derivative compound can be coated with or disposed in a selected material to protect it from natural conditions which may detrimentally effect its ability to perform its intended function. The TRH or TRH derivative compound can be administered alone, or in conjunction with either another agent as described above or with a pharmaceutically-acceptable carrier, or both. The TRH or TRH derivative compound can be administered prior to the administration of the other agent, simultaneously with the agent, or after the administration of the agent. Furthermore, the TRH or TRH derivative compound can also be administered in a proform which is converted into its active metabolite, or more active metabolite in vivo.

The language “biological activities” of TRH or TRH derivative includes all activities elicited by TRH or TRH derivative compounds in a responsive cell. It includes genomic and non-genomic activities elicited by these compounds.

The term “diabetes mellitus” is intended to have its medical meaning, namely, variable disorder of carbohydrate metabolism caused by a combination of hereditary and environmental factors and usually characterized by inadequate secretion or utilization of insulin, by excessive urine production, by excessive amounts of sugar in the blood and urine, and by thirst, hunger, and loss of weight. The term “insulin-dependent diabetes mellitus” is intended to have its medical meaning, namely severe diabetes mellitus with an early onset; characterized by polyuria and excessive thirst and increased appetite and weight loss and episodic ketoacidosis; diet and insulin injections are required to control the disease. The term “non-insulin-dependent diabetes mellitus” is intended to have its medical meaning, namely a mild form of diabetes mellitus that develops gradually in adults; can be precipitated by obesity or severe stress or menopause or other factors; can usually be controlled by diet and hypoglycemic agents without injections of insulin.

The term “effective amount” includes an amount effective, at dosages and for periods of time necessary, to achieve the desired result, e.g., sufficient to treat a pancreas-related disorder. An effective amount of TRH or TRH derivative compound may vary according to factors such as the disease state, age, and weight of the subject, and the ability of the TRH or TRH derivative compound to elicit a desired response in the subject. Dosage regimens may be adjusted to provide the optimum therapeutic response. An effective amount is also one in which any toxic or detrimental effects (e.g., side effects) of the TRH or TRH derivative compound are outweighed by the therapeutically beneficial effects.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a TRH or TRH derivative compound can include a single treatment or, preferably, can include a series of treatments. In one example, a subject is treated with TRH or a TRH derivative compound in the range of between about 0.1 to about 40 μg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a TRH or TRH derivative compound used for treatment may increase or decrease over the course of a particular treatment. An effective dose of the TRH analog of the present invention comprises an amount of the analog sufficient to reduce secondary injury by blocking or reducing the release of injurious endogenous substances. This dose is preferably administered once every three days to treat diabetes mellitus. It will be understood by those skilled in the art that the compound is administered chronically for the treatment of other metabolism disorders of diabetes. The administration of a TRH analog for diabetes treatment also includes simultaneous insulin treatment. Most preferably the effective dose of the TRH analog of the present invention is approximately 5-40 μg/kg body weight of the patient administered once every three days within the first month of serious hyperglycemia which also need insulin treatment simultaneously.

As used herein, the term “hydrate” means a compound of the present invention or a salt thereof, which further includes a stoichiometric or non-stoichiometric amount of water bound by non-covalent intermolecular forces.

The term “hyperglycemia-related malfunction” is intended to have its medical meaning, namely, a bodily malfunction which results from an excess of sugar in the blood.

The language “improved biological properties” refers to any activity inherent in a compound of the invention that enhances its effectiveness in vivo. In a preferred embodiment, this term refers to any qualitative or quantitative improved therapeutic property of a TRH or TRH derivative compound, such as reduced toxicity, e.g., reduced hypercalcemic activity.

The term “modulate” refers to increases or decreases in the blood glucose in an animal in response to exposure to a compound of the invention.

The term “obtaining” as used in obtaining the TRH or TRH analog or derivative as used herein is intended to include purchasing, synthesizing or otherwise acquiring TRH or the TRH analog or derivative.

The term “pancreas related disorder” is meant to include any condition or state that directly or indirectly has an adverse impact on pancreatic function or on cells or regions of the pancreas, thereby causing abnormally high blood glucose levels. The term includes, but is not limited to, diabetes mellitus, pancreatic islet destruction, pancreatic beta cell malfunction and hyperglycemia-related malfunction.

The terms “pancreatic islet destruction,” “pancreatic beta cell malfunction,” and “regeneration of pancreatic beta cells,” are intended to have their medical meanings. In addition, the term “regeneration of pancreatic beta cells” is intended to include restoration and/or improvement of pancreatic function.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The term “pharmaceutically acceptable salt,” is a salt formed from, for example, an acid and a basic group of a compound of any one of the formulae disclosed herein. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The term “pharmaceutically acceptable salt” also refers to a salt prepared from a compound of any one of the formulae disclosed herein having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

The term “pharmaceutically acceptable salt” also refers to a salt prepared from a compound of any one of the formulae disclosed herein having a basic functional group, such as an amino functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid.

The pharmaceutically acceptable solution includes any solution that is safe for injection or ingestion and is biologically inert so that it does not interfere with the active ingredient. The preferred pharmaceutically acceptable solution comprises an isotonic solution suitable for injection into a patient. For example, the isotonic solution may contain water, salt, and conventional ingredients such as glucose. The pharmaceutically acceptable solution may also contain purified water mixed with preservatives, flavors, colorants, flavor enhancing agents, and other additives such as sodium benzoate, methyl paraben, propylene glycol, glycerin, sorbitol, alcohol, sucrose, saccharin, menthol and citric acid.

The compounds of the invention are intended to include prodrugs. The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched alkyl ester moieties, (e.g., propionoic acid esters), alkenyl esters, di-alkyl-amino, alkyl esters (e.g., dimethylaminoethyl ester), acylamino alkyl esters (e.g., acetyloxymethyl ester), acyloxy alkyl esters (e.g., pivaloyloxymethyl ester), aryl esters (phenyl ester), aryl alkyl esters (e.g., benzyl ester), substituted (e.g., with methyl, halo, or methoxy substituents) aryl and aryl alkyl esters, amides, alkyl amides, di-lower alkyl amides, and hydroxy amides. Preferred prodrug moieties are propionoic acid esters and acyl esters. Prodrugs which are converted to active forms through other mechanisms in vivo are also included.

The language “reduced toxicity” is intended to include a reduction in any undesired side effect elicited by TRH or a TRH derivative when administered.

The term “regeneration” is intended to include the renewal, regrowth, or restoration of a body or a bodily part, tissue, or substance after injury or as a normal bodily process.

The term “subject” includes organisms which are capable of suffering from a pancreas-related disorder or who could otherwise benefit from the administration of a TRH or TRH derivative compound of the invention, such as human and non-human animals. Preferred human animals include human patients suffering from or prone to suffering from a pancreas-related disorder, as described herein. The term “non-human animals” of the invention includes all vertebrates, e.g., mammals, e.g., rodents, e.g., mice, and non-mammals, such as non-human primates, sheep, dog, cow, chickens, amphibians, reptiles, etc.

The phrases “systemic administration,” “administered systemically”, “peripheral administration” and “administered peripherally” as used herein mean the administration of a TRH or TRH derivative compound(s), drug or other material, such that it enters the patient's system and, thus, is subject to metabolism and other like processes, for example, subcutaneous administration.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

### Methods of the Invention

In one aspect, the invention provides a method for modulating blood glucose in an animal, comprising administering to an animal in need thereof an effective amount of a compound selected from the group consisting of thyrotropin releasing hormone (TRH), a TRH derivative, and a pharmaceutically acceptable salt, solvate, or hydrate of TRH or of a TRH derivative, to thereby modulate the blood glucose levels. In one embodiment, the blood glucose levels are modulated by treating or preventing a pancreas-related disorder. In one embodiment, the disorder is diabetes mellitus. In a further embodiment, the invention provides an effective treatment for metabolism disorder. In a further embodiment, the invention provides an effective treatment for glucose uptake in peripheral tissues. The peripheral tissues are identified as muscles but do not exclude other tissues or organs. In a further embodiment, the invention provides an effective treatment for the secondary effects of diabetes mellitus. In a further embodiment, the invention provides an effective treatment to increase tissue anti-apoptosis. The administration of an effective amount of TRH or a TRH analog to a patient who accepts insulin treatment or not can further be used to treat diabetes mellitus.

In one embodiment, the disorder is pancreatic islet destruction. In one embodiment, the method is pancreatic beta cell malfunction. In yet another embodiment, the method is a hyperglycemia-related malfunction.

In one embodiment, the invention provides a method, wherein the compound is TRH (1):

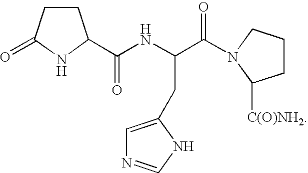

In another embodiment, the invention provides a method, wherein the compound is a TRH derivative having formula (I):

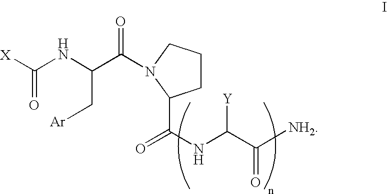

wherein:

X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;

Y is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, aryloxy, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, S(O)₂NR₂, or R;

wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;

and each X or Y may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;

Ar is selected from the following:

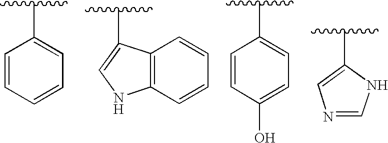

wherein each Ar group may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy; and n is an integer from 0-5.

In another embodiment, the invention provides a method, wherein the compound is a TRH derivative having formula (II):

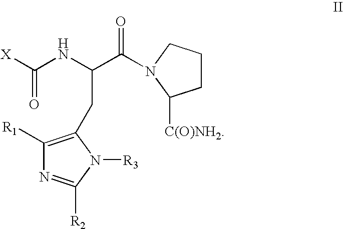

wherein:

X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;

wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;

and each X or R may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;

R₁, R₂, and R₃ are each independently, H, alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, carbonyl, carboxyl, halogen, haloalkyl, amino, aminoalkyl, thio, mercaptoalkyl, cyano, nitro, nitroso, azide, formyl, or alkylcarbonyl;

and each R₁, R₂, or R₃ is optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, aryl, heteroaryl, or aryloxy.

In another embodiment, the compound is a pharmaceutically acceptable salt, solvate, or hydrate of TRH.

In another embodiment, the compound is a pharmaceutically acceptable salt, solvate, or hydrate of a TRH derivative.

In a further embodiment, the TRH derivative is a compound wherein X is cyclyl, cycloalkyl, heterocyclyl, or heterocycloalkyl. In another embodiment, X is selected from the following:

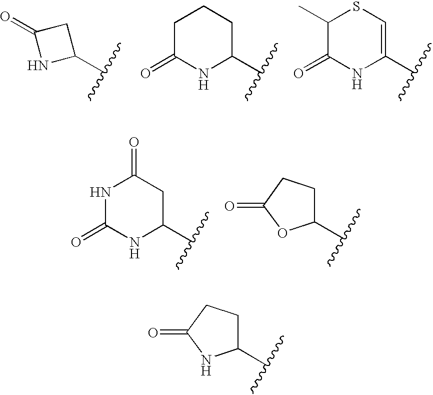

In one embodiment, the invention provides a method, wherein the compound is a TRH derivative wherein R₁ is H, alkyl, haloalkyl, halogen, or nitro. In a further embodiment, R₁ is CF₃ or I.

In another embodiment, the invention provides a method, wherein the compound is a TRH derivative wherein R₂ is H, alkyl, haloalkyl, or halogen. In a further embodiment, R₂ is CF₃ or I.

In another embodiment, the invention provides a method, wherein the compound is a TRH derivative wherein R₃ is H, alkyl, or aminoalkyl. In a further embodiment, R₃ is methyl.

In another embodiment, the invention provides a method, wherein the compound is a TRH derivative wherein R₃ is Gly such that the compound has the formula:

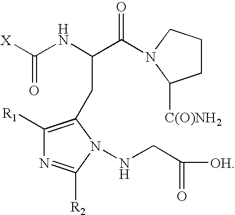

In another embodiment, the invention provides a method, wherein the compound is a TRH derivative wherein X is Gly such that the compound has the formula:

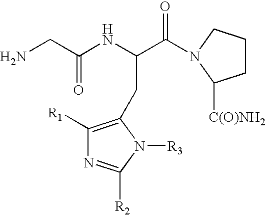

In another embodiment, the invention provides a method, wherein the compound is a TRH derivative wherein X is Leu such that the compound has the formula:

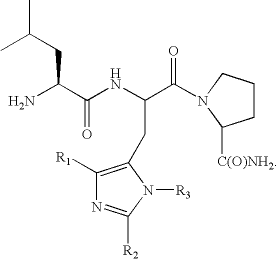

In yet another embodiment, the invention provides a method, wherein the compound is 1-{3-(1H-Imidazol-4-yl)-2-[(4-oxo-azetidine-2-carbonyl)-amino]-propionyl}-pyrrolidine-2-carboxylic acid amide (2):

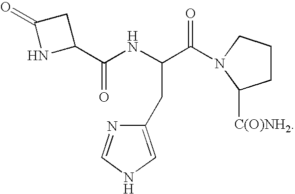

In still another embodiment, the invention provides a method, wherein the compound is 6-Oxo-piperidine-2-carboxylic acid [2-(2-carbamoyl-pyrrolidin-1-yl)-1-(1H-imidazol-4-ylmethyl)-2-oxo-ethyl]-amide (3):

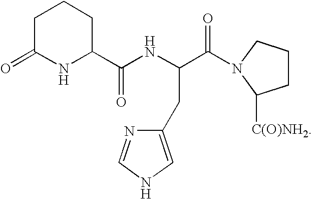

In another embodiment, the invention provides a method, wherein the compound is 6-Methyl-5-oxo-thiomorpholine-3-carboxylic acid [2-(2-carbamoyl-pyrrolidin-1-yl)-1-(1H-imidazol-4-ylmethyl)-2-oxo-ethyl]-amide (4):

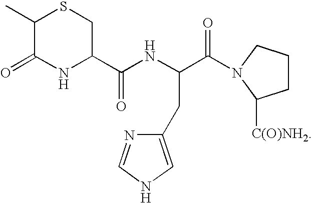

In still another embodiment, the invention provides a method, wherein the compound is 2,6-Dioxo-1,2,3,6-tetrahydro-pyrimidine-4-carboxylic acid [2-(2-carbamoyl-pyrrolidin-1-yl)-1-(1H-imidazol-4-ylmethyl)-2-oxo-ethyl]-amide (5):

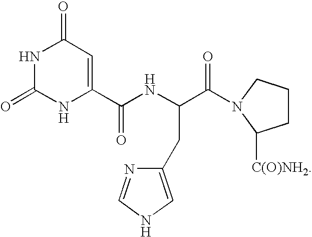

In yet another embodiment, the invention provides a method, wherein the compound is 1-{3-(1H-Imidazol-4-yl)-2-[(5-oxo-tetrahydro-furan-2-carbonyl)-amino]-propionyl}-pyrrolidine-2-carboxylic acid amide (6):

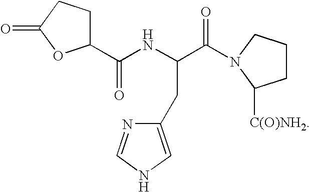

In another embodiment, the invention provides a method, wherein the compound is 1-{3-(1H-Imidazol-2,5-diiodo-4-yl)-2-[(-oxo-pyrrolidine-2-carbonyl)-amino]-propionyl}-pyrrolidine-2-carboxylic acid amide (7):

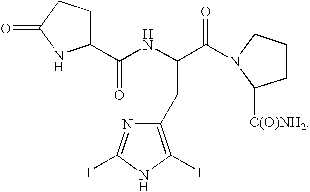

In still another embodiment, the invention provides a method, wherein the compound is (5-{3-(2-Carbamoyl-pyrrolidin-1-yl)-3-oxo-2-[(5-oxo-pyrrolidine-2-carbonyl)-amino]-propyl}-imidazol-1-ylamino)-acetic acid (8):

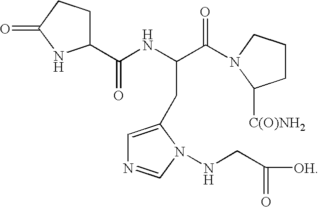

In yet another embodiment, the invention provides a method, wherein the compound is 1-[2-(2-Amino-acetylamino)-3-(3H-imidazol-4-yl)-propionyl]-pyrrolidine-2-carboxylic acid amide (9):

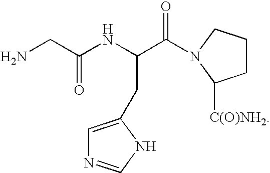

In another embodiment, the invention provides a method, wherein the compound is 1-[2-(2-Amino-4-methyl-pentanoylamino)-3-(3H-imidazol-4-yl)-propionyl]-pyrrolidine-2-carboxylic acid amide (10):

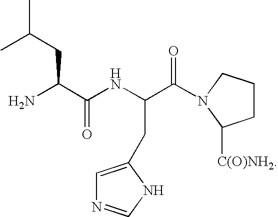

In another embodiment, the invention provides a method, wherein the compound is 1-{3-(1H-Imidazol-3-methyl-4-yl)-2-[(5-oxo-pyrrolidine-2-carbonyl)-amino]-propionyl}-pyrrolidine-2-carboxylic acid amide (“Glp-3-Me-His-Pro-NH2”) (11):

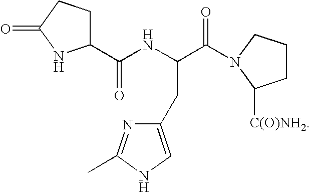

In still another embodiment, the invention provides a method, wherein the compound is 4-Amino-4-[2-(2-carbamoyl-pyrrolidin-1-yl)-1-(1H-imidazol-4-ylmethyl)-2-oxo-ethylcarbamoyl]-butyric acid (“Glu-His-Pro-NH2”) (12):

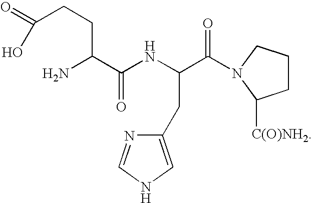

In yet another embodiment, the invention provides a method, wherein the compound is 1-{3-phenyl-2-[(5-oxo-pyrrolidine-2-carbonyl)-amino]-propionyl}-pyrrolidine-2-carboxylic acid amide (“Glp-Phe-Pro-NH2”) (13):

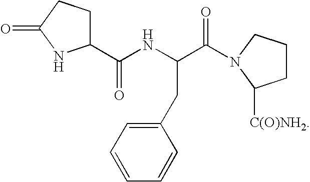

In still another embodiment, the invention provides a method, wherein the compound is (“Glp-His-Pro-Gly-NH2”) (14):

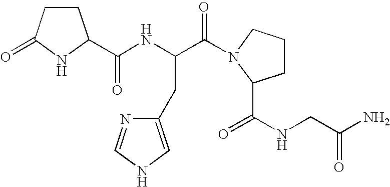

In one embodiment, the invention provides a method wherein the treated animal is a mammal. In a further embodiment, the mammal is a human.

In another embodiment, the invention provides a method which further comprises the step of obtaining the compound.

In another embodiment, the invention provides a method wherein the compound is administered orally. In another embodiment, the invention provides a method wherein the compound is administered intravenously. In another embodiment, the invention provides a method wherein the compound is administered parenterally.

In another embodiment, the invention provides a method wherein the compound is administered as a tablet, capsule, or injectable.

In one embodiment, the invention provides a method wherein the compound is administered at a concentration of 0.001 μg-100 μg/kg of body weight. In a further embodiment, the compound is administered at a concentration of about 5 μg to about 40 μg/kg of body weight.

In another aspect, the invention provides a method of regenerating of pancreatic beta cells, comprising administering to an animal in need thereof an effective amount of a compound selected from the group consisting of thyrotropin releasing hormone (TRH), a TRH derivative, and a pharmaceutically acceptable salt, solvate, or hydrate of TRH or of a TRH derivative, as recited previously. In a further embodiment, the invention provides a method of enhancing pancreatic beta cell mass, comprising administering to an animal an effective amount of a compound, as recited previously.

In one aspect, the invention provides a kit comprising a compound selected from the group consisting of thyrotropin releasing hormone (TRH), a TRH derivative, and a pharmaceutically acceptable salt, solvate, or hydrate of TRH or of a TRH derivative, together with instructions for treating a pancreas-related disorder in accordance with any of the above methods. In one embodiment, the invention provides the kit wherein the compound is formulated as a pharmaceutical composition together with a pharmaceutically acceptable diluent or carrier. In another embodiment, the invention provides the kit wherein the compound is TRH. In another embodiment, the invention provides the kit wherein the compound is a TRH derivative of formula (I):

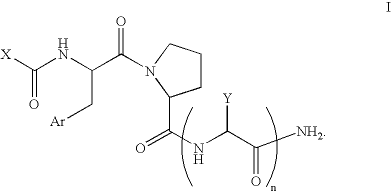

wherein:

X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;

Y is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, aryloxy, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, S(O)₂NR₂, or R;

wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;

and each X or Y may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;

Ar is selected from the following:

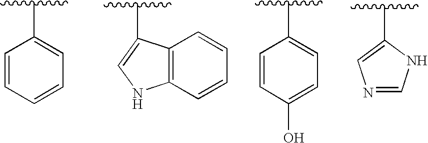

wherein each Ar group may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy; and n is an integer from 0-5.

In one aspect, the invention provides a packaged composition comprising a therapeutically effective amount of a compound selected from TRH, a TRH derivative, and pharmaceutically acceptable salt, solvate, or hydrate thereof, and a pharmaceutically acceptable diluent or carrier, wherein the composition is formulated as a pharmaceutical composition for treatment of a pancreas-related disorder, and packaged with instructions for use in accordance with any preceeding method. In one embodiment, the invention provides the packaged formulation wherein the compound is TRH. In another embodiment, the invention provides the packaged formulation wherein the compound is a TRH derivative of formula (I):

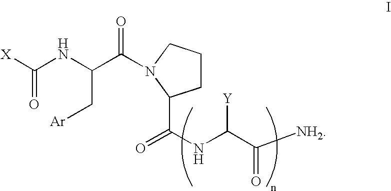

wherein:

X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;

Y is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, aryloxy, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, S(O)₂NR₂, or R;

wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;

and each X or Y may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;

Ar is selected from the following:

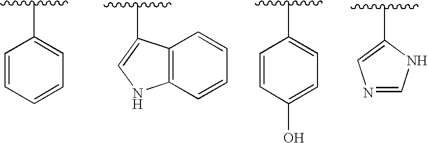

wherein each Ar group may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy; and n is an integer from 0-5.

In certain embodiments, the invention provides a packaged composition wherein the compound is administered at a concentration of 0.001 μg-100 μg/kg of body weight; preferably about 5 μg to about 40 μg/kg of body weight.

In another aspect, the invention provides a method of identifying a TRH derivative that is capable of modulating blood glucose levels comprising: (a) contacting a pancreatic cell having impaired pancreatic function with a candidate TRH derivative; and (b) determining if pancreatic function of the pancreatic cell is restored or improved, to thereby identify a TRH derivative that is capable of restoring or improving pancreatic function resulting in the modulation of blood glucose levels. In one embodiment, the invention provides a method wherein the pancreatic cell having impaired pancreatic function is within an animal. In another embodiment, the invention provides a method wherein the step of contacting comprises administering to the animal the candidate compound orally, intravenously, or parenterally. In a further embodiment, the invention provides a method, wherein the compound is TRH or a TRH derivative. In another further embodiment, the compound is TRH. In another further embodiment, the TRH derivative is a compound of formula (I):

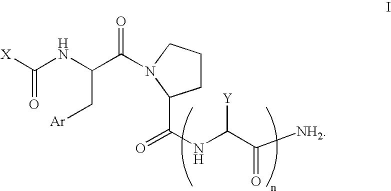

wherein:

X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;

Y is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, aryloxy, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, S(O)₂NR₂, or R;

wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;

and each X or Y may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;

Ar is selected from the following:

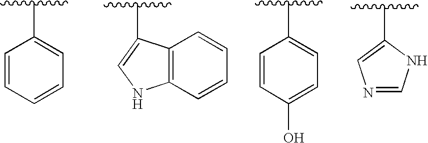

wherein each Ar group may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy; and n is an integer from 0-5.

In addition to being useful for modulating blood glucose levels, for example, in treating pancreas-related disorders, TRH and the TRH analogs of the present invention can be administered to a patient undergoing organ or cell transplant to reduce the autoimmune reaction associated with the transplant process, and to improve cell survival after transplantation. Thus, another aspect of the invention provides a method of inhibiting transplant rejection in a subject. The transplant can be any organ or cell including, but not limited to solid organs, the pancreas, pancreatic islet cells, pancreatic beta cells, and bone marrow.

### Compounds of the Invention

TRH and TRH analogs are known to exert biological activities such as acting as either a neurotransmitter or a neuromodulator or both, treating neurologic damage, including brain trauma, spinal cord trauma, neurologic damage caused by a stroke, by anesthesia or by a drug overdose, treating pancreatitis, reducing blood glucose levels, and affecting endocrine, analeptic and autonomic effects.

In the instant invention, it has been found that compounds selected from the group consisting of thyrotropin releasing hormone (TRH), a TRH derivative of formula I and formula II, and a pharmaceutically acceptable salt, solvate, or hydrate of TRH or of a TRH derivative are useful for the treatment and prevention of pancreas-related disorders, including diabetes mellitus, pancreatic islet destruction, pancreatic beta cell malfunction, and hyperglycemia-related malfunction.

Thus, in one aspect, the invention provides a novel compound wherein the compound is a TRH derivative having formula (I):

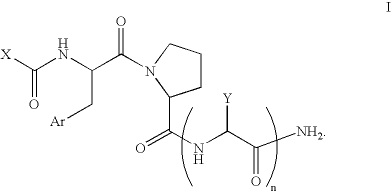

wherein:

X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;

Y is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, aryloxy, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, S(O)₂NR₂, or R;

wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;

and each X or Y may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;

Ar is selected from the following:

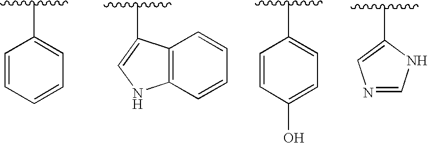

wherein each Ar group may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy; and

n is an integer from 0-5.

In one embodiment, the invention provides the novel compound wherein the compound is a TRH derivative having formula (II):

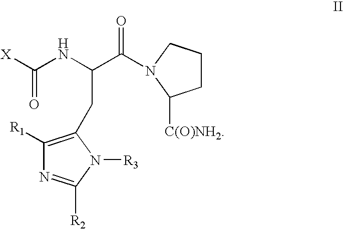

wherein:

X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;

wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;

and each X or R may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;

R₁, R₂, and R₃ are each independently, H, alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, carbonyl, carboxyl, halogen, haloalkyl, amino, aminoalkyl, thio, mercaptoalkyl, cyano, nitro, nitroso, azide, formyl, or alkylcarbonyl;

and each R₁, R₂, or R₃ is optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, aryl, heteroaryl, or aryloxy.

In another embodiment, the compound is a pharmaceutically acceptable salt, solvate, or hydrate of TRH.

In another embodiment, the compound is a pharmaceutically acceptable salt, solvate, or hydrate of a TRH derivative.

In a further embodiment, the TRH derivative is a compound wherein X is cyclyl, cycloalkyl, heterocyclyl, or heterocycloalkyl. In another embodiment, X is selected from the following:

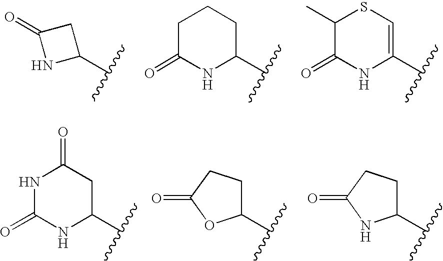

In one embodiment, the invention provides a TRH derivative wherein R₁ is H, alkyl, haloalkyl, halogen, or nitro. In a further embodiment, R₁ is CF₃ or I.

In another embodiment, the invention provides a TRH derivative wherein R₂ is H, alkyl, haloalkyl, or halogen. In a further embodiment, R₂ is CF₃ or I.

In another embodiment, the invention provides a TRH derivative wherein R₃ is H, alkyl, or aminoalkyl. In a further embodiment, R₃ is methyl.

In another embodiment, the invention provides a TRH derivative wherein R₃ is Gly such that the compound has the formula:

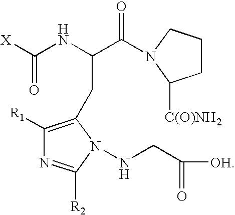

In another embodiment, the invention provides a TRH derivative wherein X is Gly such that the compound has the formula:

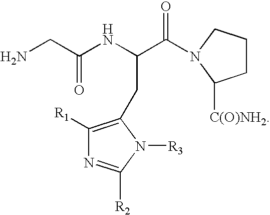

In another embodiment, the invention provides a TRH derivative wherein X is Leu such that the compound has the formula:

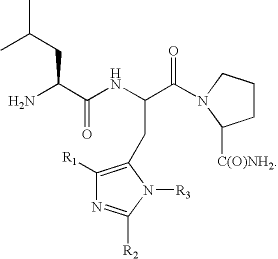

Compounds utilized in accordance with the invention include TRH (1):

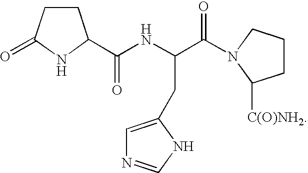

Compounds utilized in accordance with the invention also include TRH derivatives, examples of which are provided below.

1-{3-(1H-Imidazol-4-yl)-2-[(4-oxo-azetidine-2-carbonyl)-amino]-propionyl}-pyrrolidine-2-carboxylic acid amide (2):

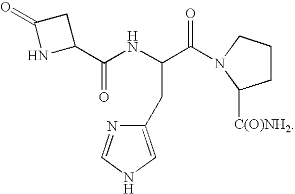

6-Oxo-piperidine-2-carboxylic acid [2-(2-carbamoyl-pyrrolidin-1-yl)-1-(1H-imidazol-4-ylmethyl)-2-oxo-ethyl]-amide (3):

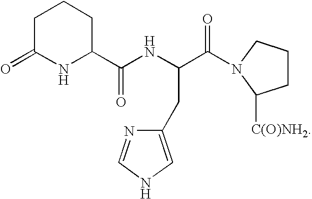

6-Methyl-5-oxo-thiomorpholine-3-carboxylic acid [2-(2-carbamoyl-pyrrolidin-1-yl)-1-(1H-imidazol-4-ylmethyl)-2-oxo-ethyl]-amide (4):

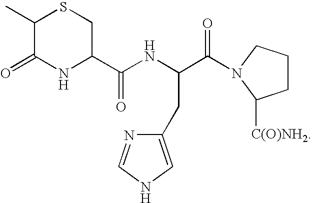

2,6-Dioxo-1,2,3,6-tetrahydro-pyrimidine-4-carboxylic acid [2-(2-carbamoyl-pyrrolidin-1-yl)-1-(1H-imidazol-4-ylmethyl)-2-oxo-ethyl]-amide (5):

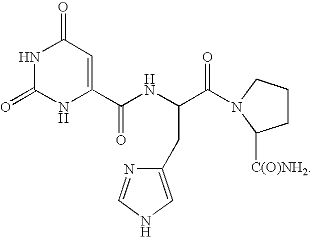

1-{3-(1H-Imidazol-4-yl)-2-[(5-oxo-tetrahydro-furan-2-carbonyl)-amino]-propionyl}-pyrrolidine-2-carboxylic acid amide (6):

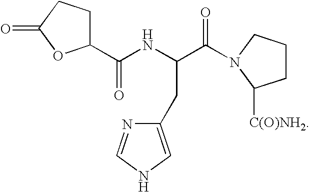

1-{3-(1H-Imidazol-2,5-diiodo-4-yl)-2-[(5-oxo-pyrrolidine-2-carbonyl)-amino]-propionyl}-pyrrolidine-2-carboxylic acid amide (7):

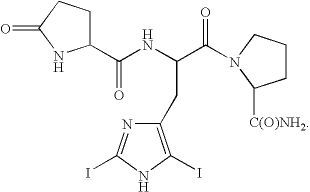

(5-{3-(2-Carbamoyl-pyrrolidin-1-yl)-3-oxo-2-[(5-oxo-pyrrolidine-2-carbonyl)-amino]-propyl}-imidazol-1-ylamino)-acetic acid (8):

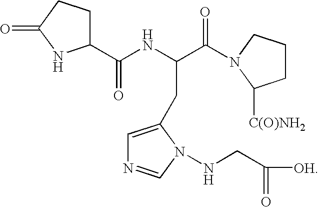

1-[2-(2-Amino-acetylamino)-3-(3H-imidazol-4-yl)-propionyl]-pyrrolidine-2-carboxylic acid amide (9):

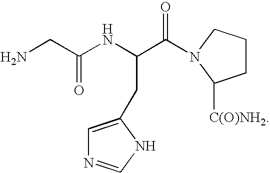

1-[2-(2-Amino-4-methyl-pentanoylamino)-3-(3H-imidazol-4-yl)-propionyl]-pyrrolidine-2-carboxylic acid amide (10):

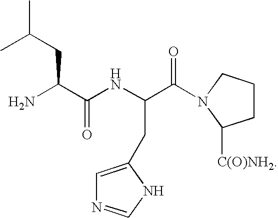

1-{3-(1H-Imidazol-3-methyl-4-yl)-2-[(5-oxo-pyrrolidine-2-carbonyl)-amino]-propionyl}-pyrrolidine-2-carboxylic acid amide (“Glp-3-Me-His-Pro-NH2”) (11):

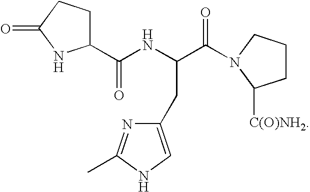

4-Amino-4-[2-(2-carbamoyl-pyrrolidin-1-yl)-1-(1H-imidazol-4-ylmethyl)-2-oxo-ethylcarbamoyl]-butyric acid (“Glu-His-Pro-NH2”) (12):

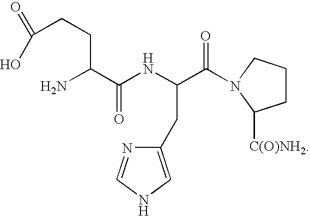

1-{3-phenyl-2-[(5-oxo-pyrrolidine-2-carbonyl)-amino]-propionyl}-pyrrolidine-2-carboxylic acid amide (“Glp-Phe-Pro-NH2”) (13):

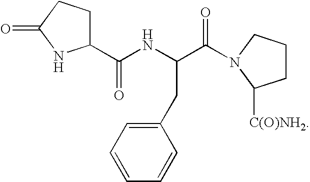

(“Glp-His-Pro-Gly-NH2”) (14):

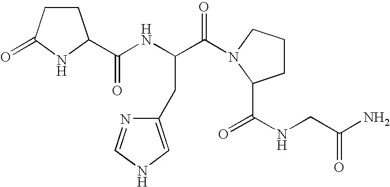

The structures of some of the compounds of the invention include asymmetric carbon atoms. Accordingly, the isomers arising from such asymmetry (e.g., all enantiomers and diastereomers) are included within the scope of the invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### Synthesis of Compounds of the Invention

Compounds of the invention can be synthesized by methods that are well known to those of skill in the art. In particular, compounds of the invention can be synthesized by methods described in this section, the examples, and the chemical literature. Examples of methods that can be used to synthesize compounds of the invention include those described in U.S. Pat. No. 5,686,420.

Certain compounds of the present invention are synthesized by starting with commercially available His analogs and modifying them in accordance with methods known to those skilled in the art. Such methods include incorporation of Gly or Leu at the N-terminal position. The various substitutions are created in accordance with the method of Labroo cited below.

Other compounds of the present invention are synthesized by starting with the thryotropin releasing hormone and modifying it in accordance with method known to those skilled in the art. The various substitutions are created in accordance with the method of Labroo cited below.

Yet other compounds of the present invention are synthesized by starting with the compound N-[[(S)-4-oxo-2-azetidinyl]carbonyl]-L-histadyl-L-prolineamide dihydrate known in the industry as YM-14673, which is available through Yamanouchi Pharmaceutical Co. LTD (Tokyo, Japan). The various substitutions are created in accordance with the method of Labroo cited below.

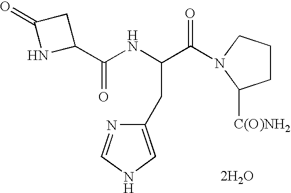

Still other compounds of the present invention are synthesized by starting with the compound orotyl-L-histidyl-L-prolineamide, known in the industry as CG 3703, which is available through Chemie Grunenethal (Stolberg, West Germany). The various substitutions are created in accordance with the method of Labroo cited below.

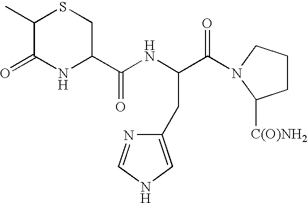

Yet other compounds of the present invention are synthesized by starting with the compound known in the industry as CG 3509, which is available through Chemie Grunenethal (Stolberg, West Germany). The various substitutions are created in accordance with the method of Labroo cited below.

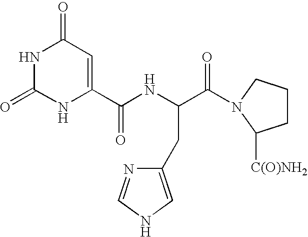

Other compounds of the present invention are synthesized by starting with the compound γ-butyrolactone-γ-carbonyl-L-histidyl-L-proline amide citrate, known in the industry as DN 1417, which is available through Takeda Chemical Industries, Ltd. (Osaka, Japan). The various substitutions are created in accordance with the method of Labroo cited below.

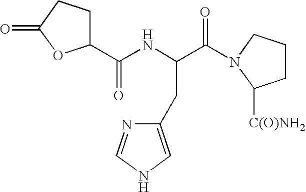

(Glp-3-Me-His-Pro-NH₂), (Glu-His-Pro-NH₂), (Glp-Phe-Pro-NH₂), and (Glp-His-Pro-Gly-NH₂), were purchased from the American Peptide Company, Inc. Other starting material peptide derivatives can be purchased or obtained from commercial sources.

The various substitutions at R¹, R², and R³ are created in accordance with the method of Labroo, V. M., Feurerstein, G., and Cohen, L. A., in “Peptides: Structure and Function” Proceedings of the Ninth American Peptide Symposium, Deber, Hruby and Kopple, eds., pp. 703-706, 1985, which is incorporated herein by reference.

For example, the incorporation of the trifluoromethyl group can be accomplished starting from Boc-His. Addition of trifluoromethyl iodide in the presence of hv provides a mixture of 2-substituted and 4-substituted Boc-CF₃-His.

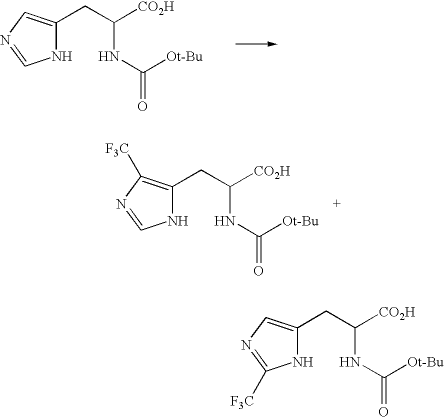

The synthesis of the tripeptide can be carried out by stepwise addition of proline under coupling conditions, followed by addition of protected PGlu and subsequent deprotection, affording the desired tripeptide. The 4-substituted variant can be synthesized in the same manner starting with the 4-substituted Boc-His.

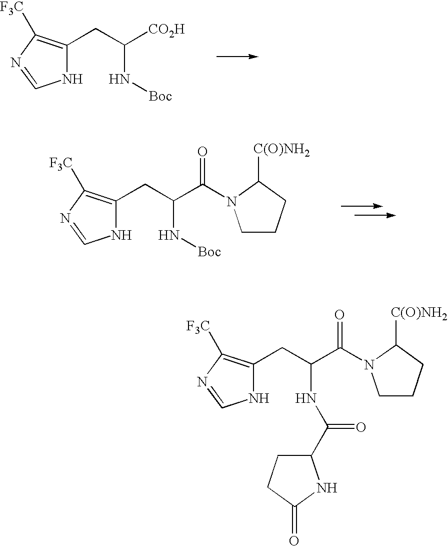

An alternative synthesis is carried out for the nitro-containing compound starting from 4-NO₂-His-OMe HCl. Addition of protected PGlu is followed by deprotection, ester hydrolysis, and Pro-NH₂ coupling.

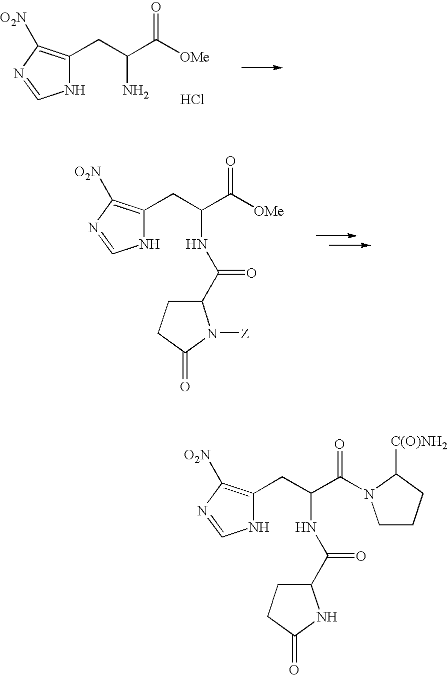

The aforementioned synthetic procedures can be used to synthesize peptide compounds of the invention.

### Pharmaceutical Compositions

The invention also provides a pharmaceutical composition, comprising an effective amount of TRH or TRH derivative of formula (I) or otherwise described herein and a pharmaceutically acceptable carrier. In a further embodiment, the effective amount is effective to treat a pancreas-related disorder, as described previously.

In one embodiment, the TRH or TRH derivative is administered to the subject using a pharmaceutically-acceptable formulation, e.g., a pharmaceutically-acceptable formulation that provides sustained delivery of the TRH or TRH derivative to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those TRH or TRH derivatives of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa buffer and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing TRH or a TRH derivative include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these compositions include the step of bringing into association TRH or a TRH derivative with the carrier and, optionally, one or more accessory ingredients. In general, the formulations are prepared by uniformly and intimately bringing into association TRH or a TRH derivative with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of TRH or a TRH derivative as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of TRH or a TRH derivative include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active TRH or a TRH derivative may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more TRH or a TRH derivative with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of TRH or a TRH derivative include powders, sprays, ointments, pastes, creams, lotions, gels, solutions, patches and inhalants. The active TRH or a TRH derivative may be mixed under sterile conditions with a pharmaceutically-acceptable carrier, and with any preservatives, buffers, or propellants which may be required.

The ointments, pastes, creams and gels may contain, in addition to TRH or a TRH derivative of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to TRH or a TRH derivative excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The TRH or a TRH derivative can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of TRH or a TRH derivative to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise TRH or a TRH derivative in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of TRH compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the TRH or a TRH derivative are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, TRH or a TRH derivative which may be used in a suitable hydrated form, and/or the pharmaceutical compositions of the present invention, are formulated into pharmaceutically-acceptable dosage forms by conventional methods known to those of skill in the art.

In certain embodiments, the invention provides a pharmaceutical composition wherein the compound is administered at a concentration of 0.001 μg-100 μg/kg of body weight; preferably about 5 μg to about 40 μg/kg of body weight.

### Exemplification of the Invention

The invention is further illustrated by the following examples which should in no way should be construed as being further limiting.

### Experimental

All operations involving TRH or TRH analogs are conducted in clear or amber-colored glassware in a nitrogen atmosphere. Tetrahydrofuran is distilled from sodium-benzophenone ketyl just prior to its use and solutions of solutes are dried with sodium sulfate. Methylene chloride is distilled over CaH₂. Melting points are determined on a Thomas-Hoover capillary apparatus and are uncorrected. Optical rotations are measured at 25° C. ¹H NMR spectra are recorded at 400 MHz in CDCl₃ unless indicated otherwise. TLC is carried out on silica gel plates (Merck PF-254) with visualization under short-wavelength UV light or by spraying the plates with 10% phosphomolybdic acid in methanol followed by heating. Flash chromatography is carried out on 40-65 μm mesh silica gel. Preparative HPLC is performed on a 5×50 cm column and 15-30 μm mesh silica gel at a flow rate of 100 ml/min.

### EXAMPLE 1

### Synthesis of 1-{3-(1H-Imidazol-2,5-diiodo-4-yl)-2-[(5-oxo-pyrrolidine-2-carbonyl)-amino]-propionyl)-pyrrolidine-2-carboxylic acid amide (7):

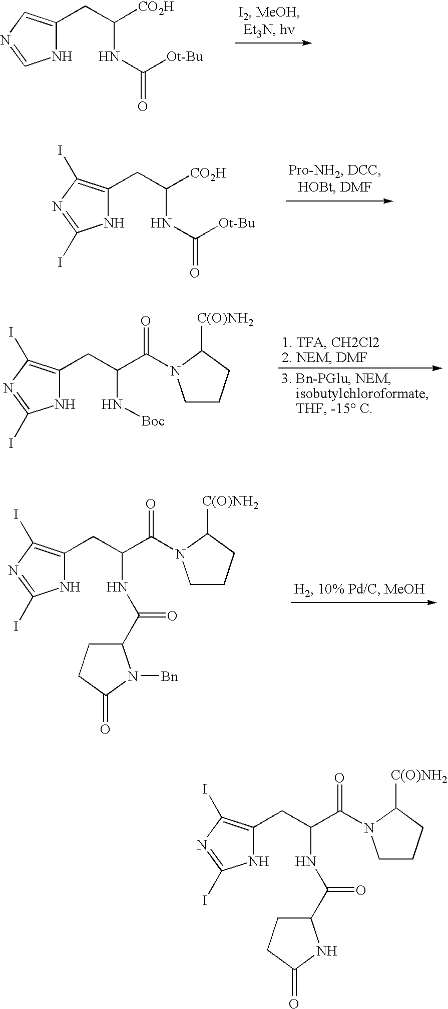

### Synthesis of 2-tert-Butoxycarbonylamino-3-(2,5-diiodo-3H-imidazol-4-yl)-propionic acid

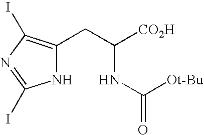

To a stirring solution of Boc-His in methanol at ambient temperature is added iodine. The reaction is subsequently subjected to hv light for 1 h. The reaction is filtered through celite, washed with methylene chloride, extracted with water, NaHCO₃ solution, dried over MgSO₄, and concentrated. 2-tert-Butoxycarbonylamino-3-(2,5-diiodo-3H-imidazol-4-yl)-propionic acid is obtained as a colorless oil and used without further purification.

### Synthesis of [2-(2-Carbamoyl-pyrrolidin-1-yl)-1-(2,5-diiodo-3H-imidazol-4-ylmethyl)-2-oxo-ethyl]-carbamic acid tert-butyl ester

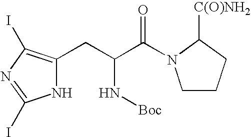

To a stirring solution of 2-tert-Butoxycarbonylamino-3-(2,5-diiodo-3H-imidazol-4-yl)-propionic acid in DMF at ambient temperature is added ProNH₂, DCC, and HOBt. After stirring for 12 h, the reaction is filtered through celite, washed with methylene chloride, extracted with water, NaHCO₃ solution, dried over MgSO₄, and concentrated. The resulting residue is purified by column chromatography to provide [2-(2-Carbamoyl-pyrrolidin-1-yl)-1-(2,5-diiodo-3H-imidazol-4-ylmethyl)-2-oxo-ethyl]-carbamic acid tert-butyl ester as a clear oil.

### Synthesis of [2-(2-Carbamoyl-pyrrolidin-1-yl)-1-(2,5-diiodo-3H-imidazol-4-ylmethyl)-2-oxo-ethyl]-carbamic acid-3-(N-benzyl)-2-pyrrolidinone

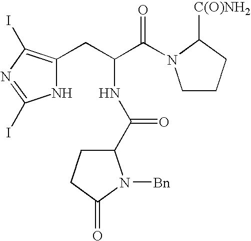

A flask is charged with a solution of [2-(2-Carbamoyl-pyrrolidin-1-yl)-1-(2,5-diiodo-3H-imidazol-4-ylmethyl)-2-oxo-ethyl]-carbamic acid tert-butyl ester in methylene chloride. To it is added trifluoroacetic acid (TFA), and the reaction is stirred for 1 h. The reaction is diluted with methylene chloride, washed with NaHCO₃ solution, dried over MgSO₄, and concentrated. The resulting residue is dissolved in THF, cooled to −15° C., and to it is added a solution of NEM in DMF, followed by Bn-PGlu and isobutylchloroformate. After stirring for 12 h, the reaction is filtered through celite, washed with diethyl ether, extracted with water, NaHCO₃ solution, dried over MgSO₄, and concentrated. The resulting residue is purified by column chromatography to provide [2-(2-Carbamoyl-pyrrolidin-1-yl)-1-(2,5-diiodo-3H-imidazol-4-ylmethyl)-2-oxo-ethyl]-carbamic acid-3-(N-benzyl)-2-pyrrolidinone as a clear oil.

### Synthesis of 1-{3-(1H-Imidazol-2,5-diiodo-4-yl)-2-[(5-oxo-pyrrolidine-2-carbonyl)-amino]-propionyl}-pyrrolidine-2-carboxylic acid amide (7)

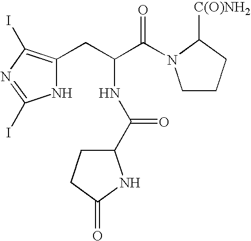

To a stirring solution of [2-(2-Carbamoyl-pyrrolidin-1-yl)-1-(2,5-diiodo-3H-imidazol-4-ylmethyl)-2-oxo-ethyl]-carbamic acid-3-(N-benzyl)-2-pyrrolidinone in methanol at ambient temperature is added 10% Pd/C. A hydrogen gas delivery apparatus is attached and the reaction is stirred for 12 h. The reaction is filtered through celite, washed with methylene chloride, extracted with water, NaHCO₃ solution, dried over MgSO₄, and concentrated to provide 7.

### EXAMPLE 2

### Restoration of Pancreatic Function Using TRH and TRH Analogs

### Materials and Methods

Male Sprague-Dawley rats (S.D. 180 g) were used in the experiments described below. The animals lived in individual metabolism cages with free access to food and water, while food and body weight were monitored daily. Peripheral blood samples (approximately 10 μl) were obtained from the tail vein of the animals. The blood glucose levels were measured by using Accu-Check Blood Glucose Meter (Roche Diagnosetics Corporation, IN). Beta cell function was evaluated once every two days over a 2-week period by an individual unaware of the treatment group. Animals were evaluated separately for blood glucose level, food intake and body weight. The endpoint check was pancreatic insulin content and pancreatic beta cell number.

The animals were anesthetized with a single dose (60 mg/kg, i.p.) of sodium pentobarbital mg/kg BW, Sigma, St. Louis, Mo.). Roger Williams Hospital Animal Welfare Committee approved the animal studies. Blood samples, pancreas and muscles were collected for further pathological and physiological tests.

Partial pancreatic tissue was extracted with 1 N glacial acetic acid for insulin assay by ELISA. Four animals were used in each group. Each pancreas was placed in head (central) to tail (peripheral) orientation and immediately snap frozen and kept at −80° C. This orientation of the tissue provided an opportunity for sectioning of equal surface areas when cutting the frozen tissue. In this manner, the pancreas sections included every portion of the pancreas in parallel, while avoiding variations caused by sectioning from different areas. The HE histological staining was performed on serial sections (5 um thick) and islets were counted in 10 slides for each sample and standardized by dividing the total area (islets/cm²).

### Chemically Induced Damage of Pancreatic Function

Streptozotocin (STZ) was used to chemically induce damage in pancreatic function in order to create a hyperglycemic state in the animals. Eight-week old male Sprague-Dawley rats (200.00 g±10 g) were injected with STZ by intraperitoneal (i.p.) (65 mg/kg body weight (BW)) to create hyperglycemic animals (24 to 48 hours). Blood glucose levels were monitored daily. Onset of hyperglycemia occurred usually about 24 to 48 hours after STZ administration.

Treatment with TRH and TRH Analogs

A group of animals were given a single dose i.p. of 20 μg/kg BW or 5-40 μg BW of TRH or TRH analogs on the seventh day or on the third day after STZ administration, which allowed for examination of the TRH/TRH analog treatment and the degree of pancreatic damage in the diabetic animal. Again, food intake, body weight and blood glucose levels of each of the animals were monitored. When hyperglycemia was determined to be reversed by TRH/TRH analog, the animal was sacrificed. Blood samples were collected for insulin assay and the pancreas and muscles were collected for further pathological and physiological tests.

Effects of Treatment with TRH and TRH Analogs

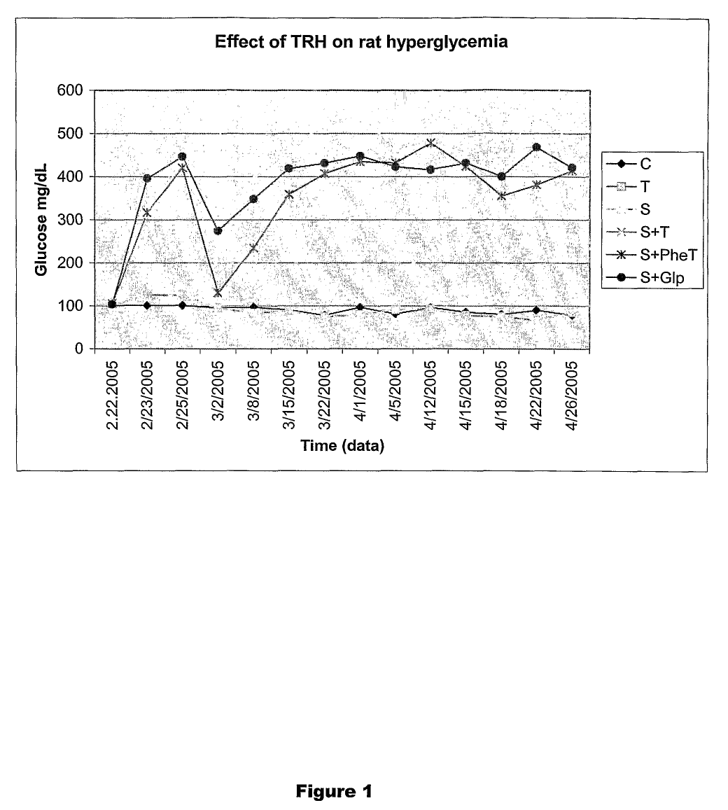

Tables 1-3 below and FIGS. 1-3 demonstrate the effect of TRH (1) and TRH analogs Glp-3-Me-His-Pro-NH₂ (compound 11 above) and Glu-His-Pro-NH₂ (compound 12 above) on hyperglycemia in rats. The figures shown in the tables are blood glucose levels (mg/dL; i.e., milligrams of glucose per deciliter of blood).

It was found that TRH normalized STZ induced hyperglycemia after seven days of TRH and STZ treatment. In particular, Table 1 shows the effect of TRH on lowering blood glucose in hyperglycemic rats. Over a two month time period, the glucose levels continually decreased when the rat was treated with TRH after STZ-induced hyperglycemia.

Additionally, TRH administration on day 3 following STZ injection also reversed the hyperglycemia after 6 days. The levels of insulin in the serum increased following TRH vs. control and STZ groups, indicating that TRH likely reversed the hyperglycemia by enhancing pancreatic beta cell function.

In further studies, it was found that TRH increases the number of pancreatic islets by activation of the TRH receptor. Composite pancreatic beta cell functions after pancreatic damage were significantly higher in the treatment group animals than in controls. TRH treatment also improved pancreatic endocrine outcome at 2 weeks after pancreatic damage.

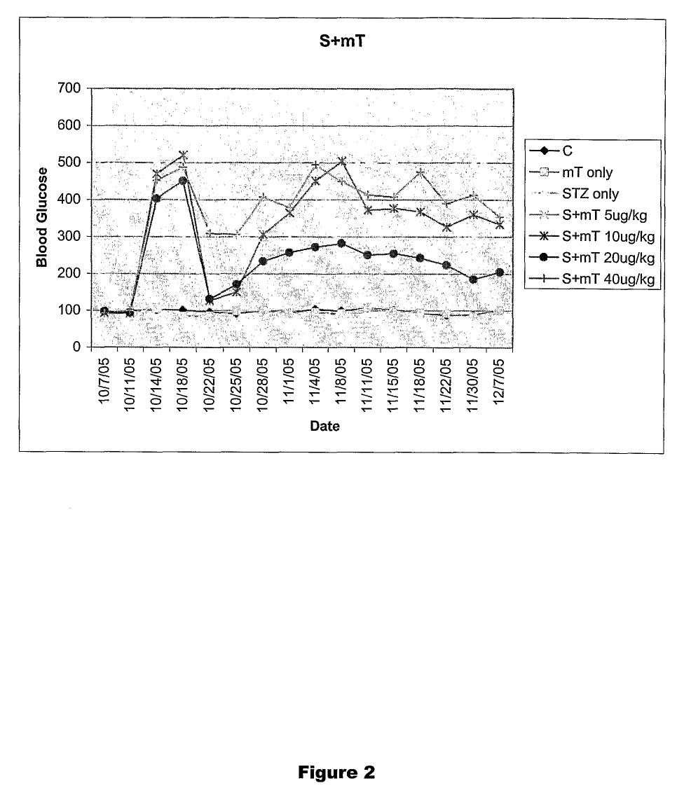

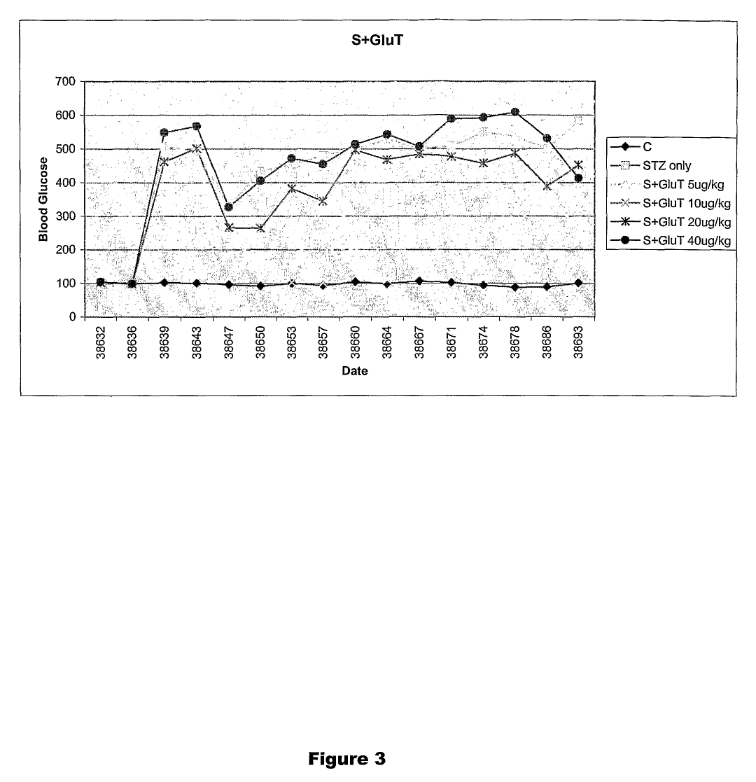

Table 2 and FIG. 2 show the relationship between mT doseage and glucose levels in STZ-induced hyperglycemic rats. Table 3 and FIG. 3 show the relationship between GluT doseage and glucose levels in STZ-induced hyperglycemic rats.

The foregoing example shows that TRH and TRH analogs, administered through intraperitoneal injection after induction of hyperglycemia significantly improve pancreatic beta cell function outcome following STZ treatment in rats.

TABLE 1
Effect of TRH (1) on rat hyperglycemia.

| | | | | | S + PheT | S + Glp |
|---|---|---|---|---|---|---|
| Day | Control | TRH (1) | S | S + TRH | (13) | (14) |
| 1 | 100.75 | 101.5 | 99.8 | 107.8 | 105.8 | 103.8 |
| 2 | 100.75 | 125 | 313.2 | 321.4 | 316.8 | 396 |
| 4 | 100.75 | 125 | 433.6 | 448.8 | 419.8 | 446 |
| 9 | 95.75 | 96 | 320 | 191.2 | 130.8 | 275 |
| 15 | 97 | 82.5 | 393.8 | 182.6 | 234.4 | 348 |
| 22 | 90.5 | 89 | 463.8 | 380.2 | 359.2 | 419 |
| 29 | 78.25 | 74.5 | 522.4 | 337 | 407.2 | 431 |
| 39 | 96.75 | 80 | 537.2 | 283.4 | 434.8 | 448 |
| 43 | 81.5 | 90.5 | 466.8 | 220.4 | 432 | 423 |
| 50 | 96 | 93 | 485 | 182 | 478 | 416 |
| 53 | 86 | 78 | 460 | 151 | 424 | 432 |
| 56 | 80 | 75 | 453 | 139 | 355 | 400 |
| 60 | 90 | 67 | 542 | 180 | 381 | 468 |
| 64 | 79 | 85 | 482 | 156 | 414 | 421 |

S = streptozotocin pancreatic beta cell killer
PheT = Glp-Phe-Pro-NH₂ (Compound 13)
Glp = Glp-His-Pro-Gly-NH₂ (Compound 14)

TABLE 2
Effect of mT (Glp-3-Me-His-Pro-NH₂ (11)) on rat hyperglycemia.

| | | | | | | S + mT | S + mT |
|---|---|---|---|---|---|---|---|
| | | | | S + mT | S + mT | 20 mg/ | 40 mg/ |
| Day | Control | mT (11) | S | 5 mg/kg | 10 mg/kg | kg | kg |
| 1 | 93 | 85 | 98 | 94 | 93 | 98 | 105 |
| 4 | 99 | 94 | 103 | 91 | 92 | 94 | 99 |
| 7 | 102 | 106 | 506 | 474 | 470 | 403 | 548 |
| 11 | 101 | 85 | 494 | 450 | 520 | 451 | 568 |
| 15 | 96 | 86 | 329 | 241 | 127 | 132 | 327 |
| 18 | 92 | 99 | 432 | 261 | 150 | 171 | 405 |
| 21 | 100 | 101 | 453 | 279 | 306 | 234 | 472 |
| 25 | 94 | 93 | 480 | 298 | 364 | 257 | 455 |
| 28 | 104 | 95 | 499 | 354 | 451 | 272 | 514 |
| 32 | 99 | 91 | 529 | 355 | 504 | 282 | 543 |
| 35 | 107 | 108 | 499 | 338 | 372 | 251 | 507 |
| 39 | 102 | 99 | 508 | 379 | 377 | 255 | 589 |
| 42 | 94 | 94 | 549 | 379 | 368 | 243 | 592 |
| 46 | 88 | 93 | 536 | 327 | 327 | 224 | 609 |
| 54 | 89 | 85 | 500 | 302 | 360 | 185 | 531 |
| 61 | 101 | 101 | 582 | 273 | 334 | 205 | 413 |

mT = 3-Me-His2 (Glp-3-Me-His-Pro-NH₂) (Compound 11)
S = STZ streptozotocin pancreatic beta cell killer

TABLE 3
Effect of GluT (Glu-His-Pro-NH₂ (12)) on rat hyperglycemia.

| | S + GluT | S + GluT | S + GluT | S + GluT |
|---|---|---|---|---|
| Day | 5 mg/kg | 10 mg/kg | 20 mg/kg | 40 mg/kg |
| 1 | 94 | 103 | 103 | 105 |
| 4 | 98 | 99 | 98 | 99 |
| 7 | 466 | 415 | 461 | 548 |
| 11 | 457 | 477 | 502 | 568 |
| 15 | 186 | 117 | 266 | 327 |
| 18 | 215 | 125 | 263 | 405 |
| 21 | 213 | 103 | 382 | 472 |
| 25 | 173 | 102 | 345 | 455 |
| 28 | 369 | 429 | 496 | 514 |
| 32 | 342 | 111 | 468 | 543 |
| 35 | 336 | 128 | 485 | 507 |
| 39 | 376 | 252 | 477 | 589 |
| 42 | 390 | 224 | 457 | 592 |
| 46 | 317 | 289 | 487 | 609 |
| 54 | 260 | 255 | 389 | 531 |
| 61 | 324 | 218 | 452 | 413 |

GluT = Glu-His-Pro-NH₂ (Compound 12)
S = STZ streptozotocin pancreatic beta cell killer

### Incorporation by Reference

The contents of all references (including literature references, issued patents, published patent applications, and co-pending patent applications) cited throughout this application are hereby expressly incorporated herein in their entireties by reference.

### Equivalents

Those skilled in the art will recognize, or be able to ascertain using no more than routine experimentation, many equivalents of the specific embodiments of the invention described herein. Such equivalents are intended to be encompassed by the following claims.

1. A method for modulating blood glucose in an animal, comprising administering to an animal in need thereof a therapeutically effective amount of a compound selected from the group consisting of thyrotropin releasing hormone (TRH), a TRH derivative, and a pharmaceutically acceptable salt, solvate, or hydrate of TRH or of a TRH derivative, to thereby modulate the blood glucose levels.

2. The method of claim 1, wherein blood glucose levels are modulated by treating or preventing a pancreas-related disorder.

3. The method of claim 2, wherein the disorder is diabetes mellitus.

4. The method of claim 2, wherein the disorder is pancreatic islet destruction.

5. The method of claim 2, wherein the disorder is pancreatic beta cell malfunction.

6. The method of claim 5, wherein the disorder is a hyperglycemia-related malfunction.

7. The method of claim 1, wherein the compound is thyrotropin releasing hormone (TRH) (1):

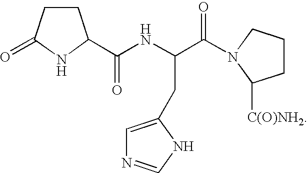

8. The method of claim 1, wherein the compound is a TRH derivative having formula (I): 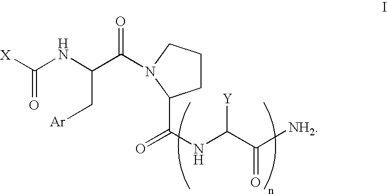
wherein:
X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;
Y is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, aryloxy, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, S(O)₂NR₂, or R;
wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;
and each X or Y may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;
Ar is selected from the following: 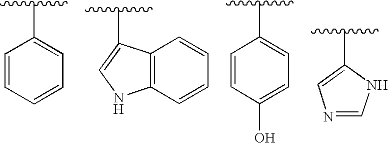
wherein each Ar group may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy; and
n is an integer from 0-5.

9. The method of claim 8, wherein the compound is a TRH derivative having formula (II): 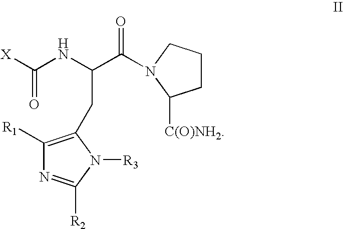
wherein:
X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;
wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;
and each X or R may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;
R₁, R₂, and R₃ are each independently, H, alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, carbonyl, carboxyl, halogen, haloalkyl, amino, aminoalkyl, thio, mercaptoalkyl, cyano, nitro, nitroso, azide, formyl, or alkylcarbonyl;
and each R₁, R₂, or R₃ is optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, aryl, heteroaryl, or aryloxy.

10. The method of claim 7, wherein the compound is a pharmaceutically acceptable salt, solvate, or hydrate of TRH.

11. The method of claim 8, wherein the compound is a pharmaceutically acceptable salt, solvate, or hydrate of the TRH derivative.

12. The method of claim 8, wherein X is cyclyl, cycloalkyl, heterocyclyl, or heterocycloalkyl.

13. The method of claim 12, wherein X is

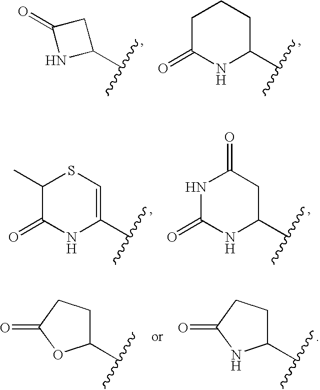

14. The method of claim 9, wherein R₁ is H, alkyl, haloalkyl, halogen, or nitro.

15. The method of claim 14, wherein R₁ is CF₃ or I.

16. The method of claim 9, wherein R₂ is H, alkyl, haloalkyl, or halogen.

17. The method of claim 16, wherein R₂ is CF₃ or I.

18. The method of claim 9, wherein R₃ is H, alkyl, or aminoalkyl.

19. The method of claim 18, wherein R₃ is methyl.

20. The method of claim 9, wherein R₃ is Gly such that the compound has the formula:

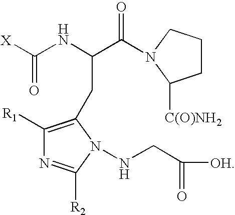

21. The method of claim 9, wherein X is Gly such that the compound has the formula:

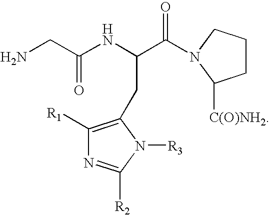

22. The method of claim 9, wherein X is Leu such that the compound has the formula:

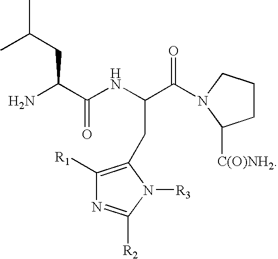

23. The method of claim 1, wherein the compound is 1-{3-(1H-lmidazol-4-yl)-2-[(4-oxo-azetidine-2-carbonyl)-amino]-propionyl}-pyrrolidine-2-carboxylic acid amide (2):

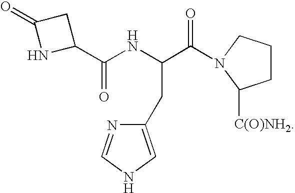

24. The method of claim 1, wherein the compound is 6-Oxo-piperidine-2-carboxylic acid [2-(2-carbamoyl-pyrrolidin-1-yl)-1-(1H-imidazol-4-ylmethyl)-2-oxo-ethyl]-amide (3):

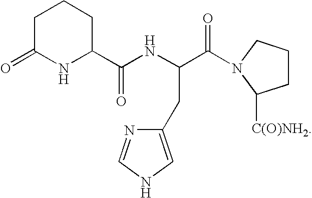

25. The method of claim 1, wherein the compound is 6-Methyl-5-oxo-thiomorpholine-3-carboxylic acid [2-(2-carbamoyl-pyrrolidin-1-yl)-1-(1H-imidazol-4-ylmethyl)-2-oxo-ethyl]-amide (4):

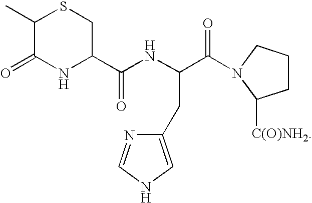

26. The method of claim 1, wherein the compound is 2,6-Dioxo-1,2,3,6-tetrahydro-pyrimidine-4-carboxylic acid [2-(2-carbamoyl-pyrrolidin-1-yl)-1-(1H-imidazol-4-ylmethyl)-2-oxo-ethyl]-amide (5):

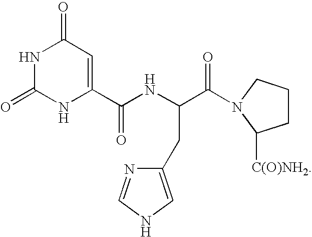

27. The method of claim 1, wherein the compound is 1-{3-(1H-lmidazol-4-yl)-2-[(5-oxo-tetrahydro-furan-2-carbonyl)-amino]-propionyl}-pyrrolidine-2-carboxylic acid amide (6):

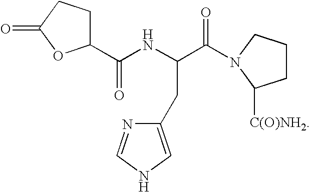

28. The method of claim 1, wherein the compound is 1-{3-(1H-Imidazol-2,5-diiodo-4-yl)-2-[(5-oxo-pyrrolidine-2-carbonyl)-amino]-propionyl}-pyrrolidine-2-carboxylic acid amide (7):

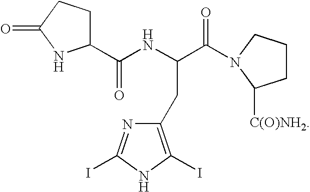

29. The method of claim 1, wherein the compound is (5-{3-(2-Carbamoyl-pyrrolidin-1-yl)-3-oxo-2-[(5-oxo-pyrrolidine-2-carbonyl)-amino]-propyl}-imidazol-1-ylamino)-acetic acid (8):

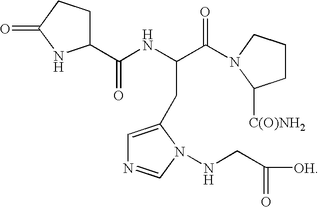

30. The method of claim 1, wherein the compound is 1-[2-(2-Amino-acetylamino)-3-(3H-imidazol-4-yl)-propionyl]-pyrrolidine-2-carboxylic acid amide (9):

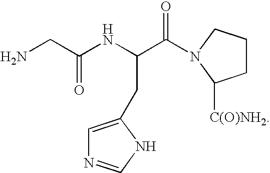

31. The method of claim 1, wherein the compound is 1-[2-(2-Amino-4-methyl-pentanoylamino)-3-(3H-imidazol-4-yl)-propionyl]-pyrrolidine-2-carboxylic acid amide (10):

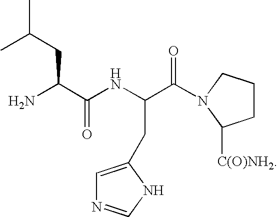

32. The method of claim 1, wherein the compound is 1-{3-(1H-lmidazol-3-methyl-4-yl)-2-[(5-oxo-pyrrolidine-2-carbonyl)-amino]-propionyl}-pyrrolidine-2-carboxylic acid amide (Glp-3-Me-His-Pro-NH2) (11):

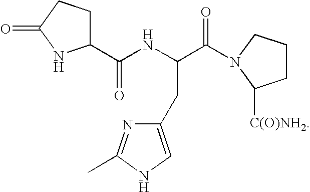

33. The method of claim 1, wherein the compound is 4-Amino-4-[2-(2-carbamoyl-pyrrolidin-1-yl)-1-(1H-imidazol-4-ylmethyl)-2-oxo-ethylcarbamoyl]-butyric acid (Glu-His-Pro-NH2) (12):

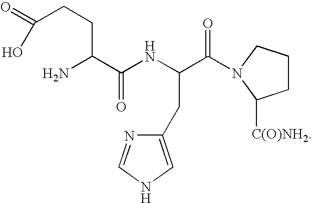

34. The method of claim 1, wherein the compound is 1-{3-phenyl-2-[(5-oxo-pyrrolidine-2-carbonyl)-amino]-propionyl}-pyrrolidine-2-carboxylic acid amide (Glp-Phe-Pro-NH2) (13):

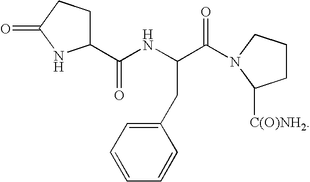

35. The method of claim 1, wherein the compound is (Glp-His-Pro-Gly-NH2) (14):

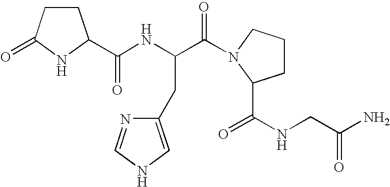

36. The method of claim 1, wherein said animal is a mammal.

37. The method of claim 36, wherein said mammal is a human.

38. The method of claim 1, which further comprises the step of obtaining the compound.

39. The method of claim 1, wherein the compound is administered orally.

40. The method of claim 1, wherein the compound is administered intravenously.

41. The method of claim 1The method of any preceeding claim, wherein the compound is administered parenterally.

42. The method of claim 1, wherein the compound is administered as a tablet, capsule, or injectable.

43. The method of claim, wherein the compound is administered at a concentration of 0.001 μg-100 μg/kg of body weight.

44. The method of claim 43, wherein the compound is administered at a concentration of about 5 μg to about 40 μg/kg of body weight.

45. A method of regenerating pancreatic beta cells, comprising administering to an animal in need thereof an effective amount of a compound recited in claim 1.

46. A kit comprising a compound selected from the group consisting of TRH, a TRH derivative and a pharmaceutically acceptable salt, solvate, or hydrate of TRH or of a TRH derivative, together with instructions for treating a pancreas-related disorder in accordance with the method of claim 2.

47-49. (canceled)

50. A packaged composition comprising a therapeutically effective amount of a compound selected from the group consisting of TRH, a TRH derivative and a pharmaceutically acceptable salt, solvate, or hydrate of TRH or of a TRH derivative and a pharmaceutically acceptable diluent or carrier, wherein the composition is formulated as a pharmaceutical composition for treatment of a pancreas-related disorder, and packaged with instructions for therapeutic use in accordance with the method of claim 1.

51-52. (canceled)

53. A method of identifying a TRH derivative that is capable of modulating blood glucose levels comprising:
(a) contacting a pancreatic cell having impaired pancreatic function with a candidate TRH derivative; and
(b) determining if pancreatic function of the pancreatic cell is restored or improved, to thereby identify a TRH derivative that is capable of restoring or improving pancreatic function resulting in the modulation of blood glucose levels.

54-56. (canceled)

57. A novel compound wherein the compound is a TRH derivative having formula (I): 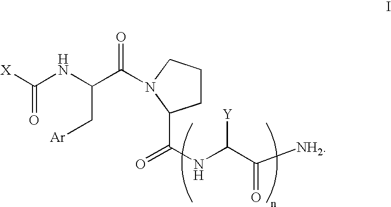
wherein:
X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;
Y is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, aryloxy, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, S(O)₂NR₂, or R;
wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;
and each X or Y may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;
Ar is selected from the following: 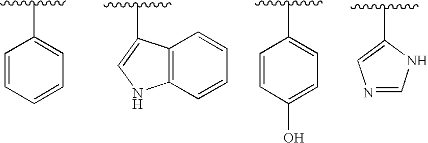
wherein each Ar group may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy; and
n is an integer from 0-5.

58. The novel compound of claim 57 wherein the compound is a TRH derivative having formula (II): 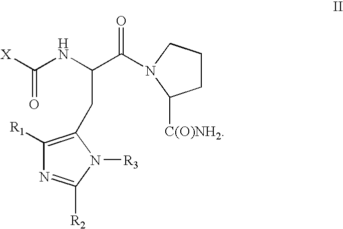
wherein:
X is alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, halogen, haloalkyl, mercaptoalkyl, cyano, nitro, nitroso, azide, OC(O)R, SO₂R, S(O)R, SR, NRCH(R)COR, NR₂, NRCOR, NRC(O)OR, NRC(O)NRR, NRSO₂ R, COR, C(O)OR, C(O)NR₂, P(O)OROR, or S(O)₂NR₂;
wherein each R is independently H, alkyl, alkenyl, alkynyl, cyclyl, cycloalkyl, heterocyclyl, heterocycloalkyl, aralkyl, heteroaralkyl, aryl, heteroaryl, alkoxy, hydroxyl, hydroxylalkyl, carbonyl, carboxyl, halogen, haloalkyl, amino, aminoalkyl, alkylamino, arylamino, thio, mercaptoalkyl, cyano, nitro, alkylcarbonyl, amido, arylsulfonyl, formyl, or aryloxy;
and each X or R may be optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, alkylamino, arylamino, alkylcarbonyl, aryl, heteroaryl, or aryloxy;
R₁, R₂, and R₃ are each independently, H, alkyl, alkenyl, alkynyl, cyclyl, cycloalkyl, heterocyclyl, heterocycloalkyl, aryl, heteroaryl, aralkyl, heteroaralkyl, alkoxy, hydroxyl, hydroxylalkyl, carbonyl, carboxyl, halogen, haloalkyl, amino, aminoalkyl, thio, mercaptoalkyl, cyano, nitro, nitroso, azide, formyl, or alkylcarbonyl;
and each R₁, R₂, or R₃ is optionally substituted with alkyl, alkoxy, hydroxyl, hydroxylalkyl, carbonyl, carboxyl, halogen, haloalkyl, amino, aminoalkyl, thio, mercaptoalkyl, cyano, nitro, formyl, alkylcarbonyl, aryl, heteroaryl, or aryloxy.

59-70. (canceled)

71. A method of inhibiting transplant rejection in a subject comprising administering to said subject a compound as recited in claim 1 in an amount effective to inhibit transplant rejection in said subject.

72-75. (canceled)
